(19) United States
(12) Patent Application Publication
(10) Pub. No.: US 2008/0009451 A1
(43) Pub. Date: Jan. 10, 2008

(54) Applications of kidney secreted bone growth factor and pharmaceutical use of flavonol and flavonol glycosides for stimulating the secretion of kidney secreted bone growth factor

(76) Inventor: Mian Long, Honolulu, HI (US)

Correspondence Address: Mian Long, 2169 Mott-Smith Dr, Honolulu, HI, 96822, US

(21) Appl. No.: 11/825,765

(22) Filed: Jul. 7, 2007

(30) Foreign Application Priority Data
Jan. 26, 2005 (CN) PCT/CN2005/000110

(51) Int. Cl. A61K 31/352; A61K 31/70; A61P 19/00; G01N 33/487

(52) U.S. Cl. 514/27; 436/63; 514/456

(57) ABSTRACT

The present invention provides a method to diagnose the kidney function of secreating kidney secreted bone growth factor (KSBGF) by examining the blood concentration of KSBGF, a method to diagnose the state of bone formation by examining the blood concentration of KSBGF, a method to produce KSBGF, a method of using KSBGF to promote bone growth, a method for screening medicine for bone reformation, a method of using flavonol and flavonol glycosides to promote renal epithelial cell proliferation, and the application of flavonol and flavonol glycosides to improve renal function, to treat renal failure, and to promote bone formation.

### FIELD OF THE INVENTION

Based on the discovery of that renal tubular cell produces kidney secreted bone growth factor (KSBGF) and flavonol and flavonol glycosides promote renal tubular cell produces KSBGF, the present invention is directed to a process to determine the quantity of KSBGF in the body fluids for diagnosing the capacity of kidney secretes KSBGF and to predict the state of bone formation, a process for using KSBGF to promote bone formation, a process for screening for medicinal treatment designed to promote bone formation, a method to stimulate kidney to secrete the kidney secreted bone growth factor (KSBGF), a method for using flavonol and flavonol glycosides to promote renal tubular cell proliferation and the use of flavonol and flavonol glycosides for treating kidney deficiency and promoting bone formation.

### BACKGROUND OF THE INVENTION

The role of the kidney in regulating bone formation can be seen in the patients with renal failure and associated secondary osteoporosis. In human and mammals, bones are continuously remodeled through repeated cycles of destruction and rebuilding (Science 1 September 2000; 289: 1497). Weak bone formation following by the renal failure has been seen in clinical studies, which is not recoverable by supplementing with vitamin D and calcium (Kanis J A., Vitamin D analogs: from renal bone disease to osteoporosis, Kidney Int Suppl. December 1999; 73: S77-81). Whether the regulatory aspects of bone formation and depletion involve one or several, small or large molecules from kidney is unclear, but collectively they are termed kidney secreted bone growth factor(s) (KSBGF).

There is significant precedence for using herbal medicine for treating renal failure in Traditional Chinese Medicine. The theory that the “kidney controls the bone” was recorded in traditional Chinese medicine. Practitioners observed that the kidney regulates bone metabolism, since the condition of patients with kidney deficiency was often accompanied by osteoporosis. Furthermore, the herbal medicine that believed tonic for kidney also promotes the bone formation in patients with fracture. But the active components present in the herbs and their principle effective components were unclear. It has not been observed in the literature that the kidney could secrete bone growth factor and in turn increase bone formation.

1. The plant, Drynaria fortunei (Kunze, J. Smith), which goes also by the names Rhizoma Drynariae (botanical name) and Gu Sui Bu (Chinese name), and termed here GSB, is traditionally used as a stimulant for bone formation with fracture (Bensky D and Gamble A. Chinese Herbal Medicine Materia Medica. with ten kaptchuk. Illustrations adapted 1986 ISBN 0-939616-15-7 p 350.). Modern research also has confirmed the ability of GSB to promote bone formation (Wong R W, Rabie A B. Systemic effect of crude extract from rhizome of Drynaria fortunei on bone formation in mice. Phytother Res. April 2006; 20(4):313-5) and to increase the absorption of ⁴⁵Ca by bone in a fractured-femur mouse model (Wang. Zhiyu. Effect of Gu Sui Bu on the fracture recovery. Bulletin of Bejing university of Chinese Traditional Medicine, 1980, 3 (3) 13). A flavonoid fraction of GSB was been shown to increase significantly bone density in patients with osteoporosis (Gu. M, Ge J N. Gu-Sui-Bu on treating primary osteoporosis. Chinese Rehabilitation. 19 (5): 297, November 2004). In our laboratory, the flavonoid fraction of GSB was isolated and demonstrated to have a protective effect against gentamicin- and mercuric chloride-induced renal toxicity, and to prolong the survival time of nephrectomized animal by acting on the regeneration of tubular cells (Long M. Qiu D. Li F. Johnson F. et al. Flavonoid of Drynaria fortunei protects against acute renal failure. Phytotherapy Research. 19(5): 422-7, May 2005). Kaempferol was later isolated and identified from the same flavonoid fraction of GSB (unpublished data).

Bone Morphogenic Proteins (BMPs) are secreted proteins that play an important role in bone development and maturation. There are 15 BMP and two BMP receptors (BMPR I and BMPR II) which have been identified so far (Orthopedic Clinics of North America. 33(2):447-63, ix, April 2002). Reddi et al. also reported that BMPs play an important role on the bone formation in healing fracture (Journal of Bone & Joint Surgery—American Volume. 83-A Suppl 1(Pt 1): S1-6, 2001). Lund et al.'s experiments showed the benefit of BMP7 on the osteoporosis associated with renal failure in animal models (Journal of the American Society of Nephrology, 15(2):359-69, February 2004). Above examples confirmed the effects of BMP on bone metabolism. BMP-7 has been demonstrated to prevent renal failure in a variety of animal models of renal injury including acute renal ischemia, unilateral uretheral obstruction, diabetic nephropathy, and lupus nephritis (J Clin Invest 1998; 102:202-214. J Am Soc Nephrol 2002; 13:S14-S21. Kidney Int 2003; 63:2037-2049. Nature Med 2003; 9:964-968). BMPs were also found to be expressed by several different cell lines, such as glomerular, mesangial, epithelial, endothelial cells, and distal tubular MDCK cells (a dog kidney cell line). However, expression by human kidney (HK-2) cells was not found (Journal of Cellular Physiology. 181(3):410-5, December 1999). The mechanism of producing, secreting and transferring of BMPs in human is unclear.

So far, the method for diagnosing the early stage of deficiency of renal function is lack. The mechanism of the regulation of bone formation by kidney function is not clear. Therefore, medicine for promoting kidney function and bone formation is lack. This invention provides a method for early diagnosis of kidney deficiency and the risk of osteoporosis. It also provides the medicine for treating deficiency of kidney function or renal failure, and also for promoting bone formation.

### SUMMARY OF INVENTION

One aspect of the present invention is directed to a method of examining the function of kidney, that includes the process of examine the level of KSBGF in body fluids and the process of analyzing the level of kidney function by the level of KSBGF in the body fluid.

One aspect of the present invention is directed to a method of examine the state of bone formation and bone metabolism, that includes the process of examine the level of KSBGF in body fluids and the process of analyzing the state of bone formation and metabolism by the level of KSBGF in the body fluid.

One aspect of the present invention is directed to a process for promoting renal tubular cell proliferation that includes the process of applying flavonol and flavonol glycosides to stimulate tubular cell proliferation.

One aspect of the present invention is directed to a process for promoting bone formation that includes the process of applying flavonol and flavonol glycosides to stimulate tubular cells secret KSBGF and the process of KSBGF contact with the bone cells.

An additional aspect of the invention is directed to a method for producing KSBGF. In particular, it is to provide a process for renal tubular cell secreting KSBGF and/or a process for renal tubular cells contact with flavonol and flavonol glycosides.

Another aspect of the invention is directed to a process for screening the medicine for promoting bone growth. In particular, the invention is directed toward a process for the candidates contacting with renal tubular cells and examine the concentration of KSBGF.

A further aspect of the invention is to provide a process for screening the medicine for treating the disease based on bone growth disorder. In particular, the invention includes a process for the candidates contacting with renal tubular cells and examine the producing of KSBGF.

A further aspect of the invention is to provide a process for using KSBGF to produce the medicine for improving bone formation.

In this invention, the flavonol includes one or more than one of the following flavonol: Datiscetin, Fisetin, Kaempferol quercetin tangeretin, nobiletin, azeleatin 5-deoxykaempferol galangin glepidotin Gossypetin, herbacetin, 6-hydrykaempferol isoquercitrin isorhamnetin kaempferide morin myricetin patuletin, quercetagetin rhamnetin robinetin and Seangularetin. The better choice is fisetin, Kaempferol, quercetin etc. The flavonol glucoside includes one or more than one of the below flavonol: icariin gossypin, rutin hyperin myricitrin kaempferitrin Fesetin 8-c-glycoside quercitrin, quercimeritrin and robinetin. The advanced choice is icariin.

The present invention is to provide an application of flavonol and flavonol glycosides in producing medicines for treating deficiency in kidney function and renal failure.

The present invention is to provide an application of flavonol and flavonol glycosides in producing medicines for kidney deficiency and promoting bone formation and metabolism.

### BRIEF SUMMARY OF THE FIGURES

FIG. 1. Shows the structure of flavonol, flavonol glycosides and non-flavonol control. Apigenin and isokaempferol, which without 3-OH, are not flavonol. Flavonol is one type of flavonoid with a 3-OH. Flavonol glycosides are flavonoid with a 3-O-glycoside.

FIG. 2A shows that flavonols and flavonol glycosides increase opossum kidney (OK) cell proliferation. Apigenin did not increase OK cell proliferation. The higher optic density (OD), the higher cell proliferation

FIG. 2B Shows isokaempferol did not increase OK cell proliferation compared with kaempferol.

FIG. 3A shows flavonol and flavonol glycosides conditioned OK cell culture-medium (OKM) increased osteoblast proliferation with dose response manner.

Isokaempferol and Apigenin conditioned OKM did not increase osteoblast proliferation.

FIG. 3B shows flavonol and flavonol glycosides, apigenin and isokaempferol conditioned OK cell culture medium do not increase human heard fibroblast proliferation.

FIG. 4A shows the effect of kaempferol on kidney cell, osteoblast and fibroblast cell proliferation. Kaempferol at various concentrations only increased OK cell proliferation, but not osteoblast and fibroblast.

FIG. 4B shows the process of preparation of OK cell conditioned-culture medium (OKM) with various doses of kaempferol. The kidney cell growth was examined after 24 hours incubation. The control medium (CM) was prepared at the same time and with the same condition of OKM but without cells. Both OKM and CM were used for MC 3T3 E1 cell culture.

FIG. 4C shows osteoblast growth in OKM treated with various concentrations of kaempferol (from FIG. 4B). The data show ₀OKM significantly increased the osteoblast proliferation compared with CM. Kaempferol conditioned-OKM increasing osteoblast proliferation with a dose response manner. All the dada together indicate that kidney cells secrete KSBGF, which in turn promotes bone formation. Kaempferol stimulates kidney cells increase the secretion of KSBGF.

FIG. 5A shows HFF cell growth in OKM, CM and fresh medium under the condition with and without kaempferol. HFF cell grow in OKM was similar to that in CM and fresh medium. OKM does not promote HFF cell proliferation.

FIG. 5B: shows ₀OKM significantly increased osteoblast cell proliferation and ₇₀ₖₐₑOKM additional increased osteoblast cell proliferation. The CM and fresh medium both with and without kaempferol did not increase human fibroblast proliferation.

FIG. 6A shows the results of immunostain on OK cell by using anti-BMP receptor II and I. Although BMPR I was not found on OK cells, BMPR II was found on OK cells. Furthermore, the presence of kaempferol increased the density of BMPR II on OK cells.

FIG. 6B shows the results of Western Blot analysis of OK cell by using anti-BMPR I and II. BMPR I was not found in OK cells sample, BMPR II was found on OK cell. Similarly, the presence of kaempferol in the medium increased the quantity of BMPR II on OK cells. Both FIGS. 6A and 6B indicate that kaempferol increases the quantity of BMPR II on OK cell.

FIG. 7 shows the quantity of BMP 2/4 and 7 in the kaempferol conditioned-OKM. BMP7 was increased in dose response to kaempferol concentration in the OK cell medium (FIG. 7A), as was BMP2/4 (FIG. 7B). FIG. 7C is the over exposed film of FIG. 7B. There is a protein with the molecular weight about 63 KDa (P63) found on the film. This quantity of P63 also increased with the dose of kaempferol.

FIG. 8 shows that, MC 3T3 E1 cell grown in the ₀HKM is significantly increased than in the ₀CM. MC 3T3E1 cell grown in 70 nmHKM is significantly increased than in the ₇₀ₙₘCM. MC 3T3 E1 cell grown in the _{70 nm}HKM is also significantly increased than in the ₀HKM. All together shows that HKM promoted MC 3T3 E1 proliferation and kaempferol additionally increases the promotion. The data indicates that human kidney tubular cells secrete KSBGF, which in turns to promote osteoblast proliferation and kaempferol increases the secretion.

FIG. 8 also shows that, HF cell grown are similar in the _{70 nm}HKM, ₀HKM, _{70 nm}CM and ₀CM. Both 70nmHKM and ₀HKM showed no promoting effect on the HF proliferation. The action of human kidney tubular cell secreted KSBGF to promote osteoblast proliferation is restricted.

### DETAILED DESCRIPTION OF THE INVENTION

In this invention, the flavonol is designated that there is 3-OH on the flavones. The flavonol glycosides are designated that there is 3-o-aglycone or aglycones. In this invention, the flavonol include, but not only include: datiscetin, fisetin, kaempferol quercetin tangeretin, nobiletin, azeleatin 5-deoxykaempferol galangin glepidotin A gossypetin, herbacetin, 6-hydrykaempferol isoquercitrin isorhamnetin kaempferide morin myricetin patuletin, quercetagetin rhamnetin robinetin and seangularetin. The advanced choice is fisetin, kaempferol, quercetin etc. The flavonol glycosides include, but not only include: icariin gossypin, rutin hyperin myricitrin kaempferitrin Fesetin 8-c-glycoside quercitrin, quercimeritrin and robinetin. The preference is icariin.

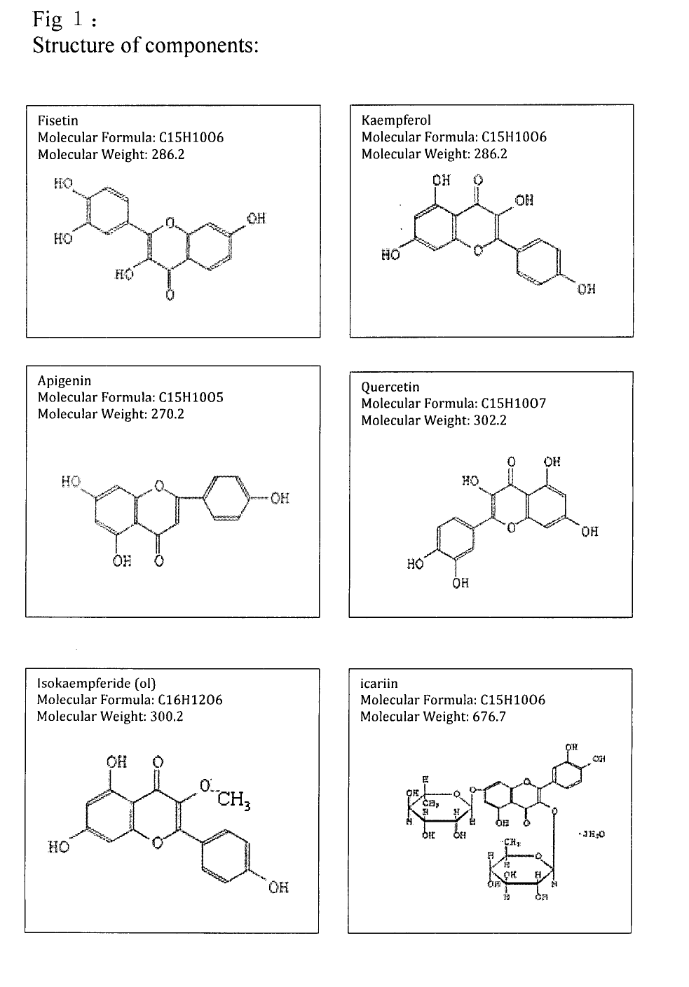

The flavonol showed on the FIG. 1 are the preference choice of flavonol.

This applicant excitedly found that renal tubular cells secrete KSBGF. It implies that the kidney is the organ that secretes KSBGF. In addition, this applicant found that flavonol and flavonol glycosides stimulate renal tubular cells secreted significantly more KSBGF. This further confirms that the kidney is the organ that secretes KSBGF.

In this invention, the concepts of KSBGFs are the factors which are secreted from the kidney and which stimulates bone formation. These include but are not limited to BMP 2, 4, 7, 8, 9 and 10.

Based on practicing scheme, this invention is to provide a process for promoting renal tubular cell proliferation. It includes the above process of flavonol and flavonol glycosides come in contact with renal tubular cells. More specifically, the appropriate condition of the medium for kidney cell growth is allowed to be chosen.

Based on another practicing scheme, this invention is directed to a process for producing KSBGF, which includes the processing of renal tubular cell secreted KSBGF. As discussed above renal tubular cells secrete KSBGF, therefore, to culture renal tubular cells, which in the right condition with right medium may produce KSBGF. In this aspect, this invention includes the process of the renal tubular cells coming in contact with flavonol and flavonol glycosides. This means adding flavonol or flavonol glycosides into culture medium to increase the secretion of KSBGF. Based on this invention, the KSBGF can be isolated and identified from the medium.

Based on another practicing scheme, this invention is directed to a process for using KSBGF for promoting bone formation.

Based on another practicing scheme, this invention is directed toward a process for screening the medicine for promoting bone growth and treating bone growth diseases, Furthermore, it includes the process to administer the candidates to in contact with renal tubular cells, then examine the level of KSBGF. In detail, culture the candidate in the possible condition of medium with renal tubular cells and then examine the concentration of KSBGF. The control sample will be the same condition but without testing sample. The active component will increase the KSBGF after incubation.

Based on practicing scheme, this invention is directed toward a method for treating renal failure. It includes the process of administrating the effective dose of flavonol or flavonol glycosides to the patients.

Based on another practicing scheme, this invention is directed toward a process for promoting bone formation and treating disease of bone formation disorder, which includes administrating the effective dose of Flavonol and Flavonol glycosides to patients. Based on this process, flavonol and flavonol glycosides can be used for treating bone diseases such as osteoporosis.

Said flavonol and flavonol glycosidess are used individual and a combination of more than one of the components.

The term “effective dose” indicates the dose of the medicine which effect on the individual. The exact doses depend on the goal of the treatment which may be determined by professionals using the known methods. It depends on the methods of the medication: orally or externally, different location, patient's status, age, body weight, sex, health status, dietary habit, time on the medication, the interaction with other medications, and the severity of the disease. It also can be determined by professionals in this field using the conventional experiments.

The term “patient”, in this invention, includes human and animal (especially the mammals) and other organisms. Therefore, the medicine included in this invention can be useful for human and animal. The advanced choice of the “patient” is mammal. The most advanced choice of the “patients” is human.

The medication in this invention can be administrated in various ways, but not limited to: orally, subcutaneous, intravenous, intranasal, intracutaneous, intraperitoreal, intramuscular, intrapulmonary, intravaginal, intrarectal or intraocular, etc.

The formulation of flavonol or flavonol glycosidess in this invention can further contain at least one other ingredient from the source of flavonol and flavonol glycosides. The medical used formulations of flavonol and flavonol glycosides in this invention can further contain at least one ingredient which selected from a filler, a binder, a disintegrant, a suspending agent, a coating agent, a sweetener, a flavoring, a lubricant, dilutions colorants polymer wax or other ingredient which described in Handbook of Pharmaceutical Excipients, 2ⁿ Edition, American Lachman, Leon, 1976; Pharmaceutical Dosage Forms: Tablets Volume 1, 2^{nd} Edition, Lieberman, Herbert A., et al, 1989; Modern Pharmaceutics, Banker, Gilbert and Rhodes, Christopher T, 1979; and Remington's Pharmaceutical Sciences, 18^{th} Edition, 1990, each of which is incorporated herein by reference in its entirety.

The medical used formulations of flavonol or flavonol glycosides, in this invention, can further contain at least one preservatives, stabilizers, anti-oxidants, silica flow conditioners, antiadherents or glidants.

The formulation of flavonol and flavonol glycosides in this invention can further be formed to be pills, capsules liquids, patches, injections or formulae for using for nose or eye.

### EXAMPLE

The examples listed below explained this invention. They are not limit of this invention.

All of the culture medium Minimum essential medium (MEM) (11095), MEM non-essential amino acids solution (11140), sodium pyruvate solution (11360) and FBS were purchased from Gibicol.

Except when otherwise indicated, opossum kidney tubular cells were used as the kidney cell line. These cells were obtained from American Type Culture Collection (ATCC), Manassas, Va. Osteoblasts used MC 3T3 E1 cells from Technologies Grang Island, N.Y. The fibroblast used here were human heart fibroblast from ATCC. All antibodies were from Santa Cruz Biotechnology Inc.

### Example 1

Flavonol Increased Renal Tubular Cell Proliferation Ok cells at 1×10⁴/ml (0.1 ml) were added into 96 wells plate. Several flavonol [symbol] Fisetin, [symbol] Kaempferol, [symbol] quercetin a [symbol] [symbol] icariin or the control flavonoid (which are not flavonol) such as apigenin and kaempferol-3-methyl ether were added to make the final concentration at 0-60 nM. (0-79 nM)

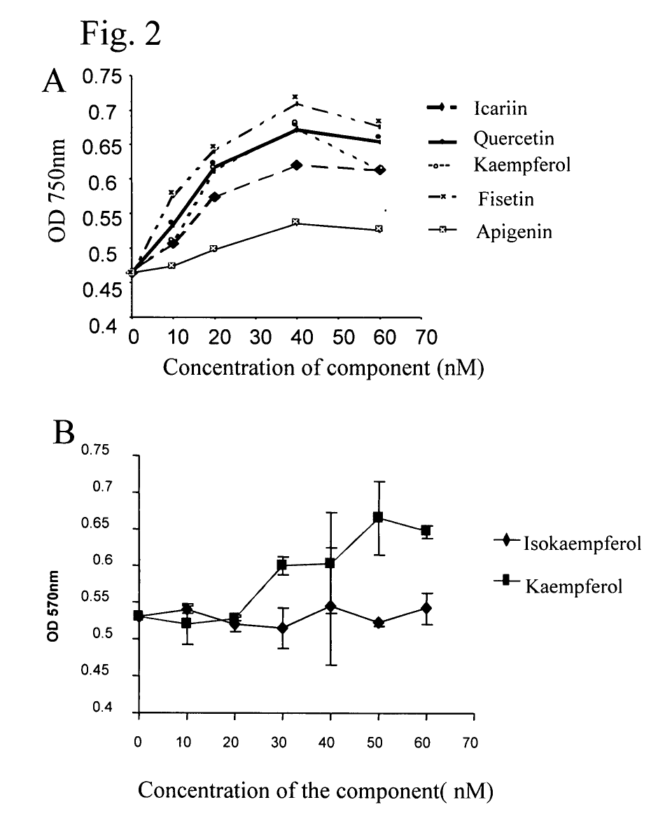

After three days incubation, the cell growth was examined with CellTiter 96 (Non-Radioactive Cell Proliferation Assay kit) (Promega) at OD570 nM. The higher OD, the higher the numbers of living cells. (FIG. 2.)

FIG. 2A shows Flavonol and flavonol glycosides significantly increased kidney tubular cell proliferation, but non-flavonol component, apigenin and kaempferol-3-methyl ether, showed no effect.

The structure of apigenin and kaempferol-3-methyl are similar to flavonol, but the 3-OH on either one was absent or blocked by a —CH3. This indicates that 3-OH or 3-0-glycoside of flavonol is the functional group of promoting renal tubular cell proliferation.

Therefore, this invention indicates that flavonol and flavonol glycosides can be used for treating renal failure and improving kidney function.

### Example 2

Flavonol and Flavonol Glycosides do not Directly Stimulate Osteoblast Proliferation, but the OKM and the Flavonol Conditioned OKM Promote Osteoblast Proliferation.

In this example, OK cells were used as the kidney tubular cell. OK cells at 6×10⁴ml (3 ml) were added into 6 wells plate and various concentrations of flavonol, flavonol glycosides, apigenin and isokaempferol were applied. At the same time, the control culture medium (CM) with the same condition as above but without cells was prepared. After 24 hours incubation, the medium was collected for osteoblast culture. The OK cell growth was examined as discussed above.

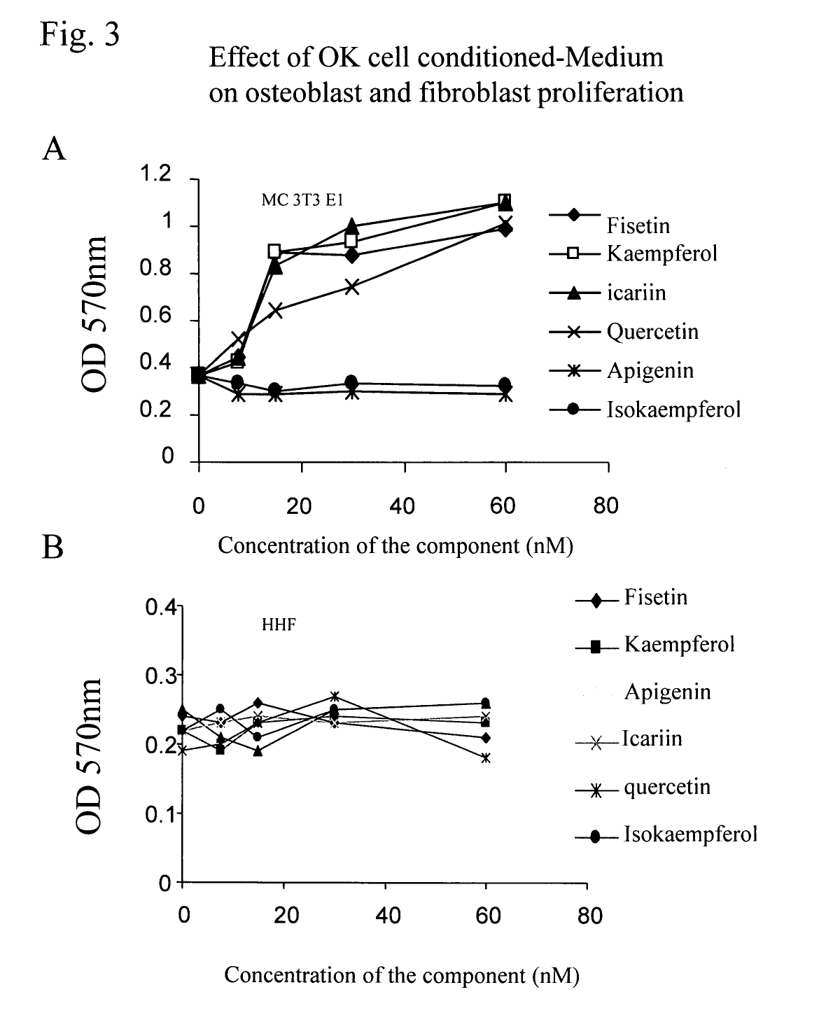

In this example, the osteoblast was the MC 3T3 E1 cell. MC 3T3 E1 at 1×10⁵/ml (20 μl) was added into 96 wells plate. Additionally, 80 μL of OKM or CM were recruited. After 3 days incubation, the osteoblast growth was examined as discussed above. The results show on FIG. 3.

FIG. 3 shows that MC 3T3 E1 cell growth in the OKM was significantly increased compared with those in the CM. MC 3T3 E1 cell growth in the flavonol or flavonol glycosides conditioned-OKM was more increased than in the control OKM. MC 3T3 E1 growth in the non-flavonol conditioned-OKM was not increased compared to the control.

FIG. 3B shows that there is promoting effect of flavonol on the MC 3T3 E1 cell growth.

The above data indicate that OKM stimulates osteoblast proliferation. However, the flavonol and flavonol glycosides treated OKM more increased osteoblast proliferation than control OKM.

More specifically, based on the findings that renal tubular cells secrete KSBGF, this invention is directed toward a process to examine the concentration of KSBGF in body fluid for diagnosing the kidney function of the secretion of KSBGF. Furthermore, the invention also is directed toward a process to examine the concentration of KSBGF in the body fluid for prognosticating the state of bone formation. The renal tubular cells are able to be used for producing KSBGF. KSBGF is able to be used for promoting osteoblast proliferation. Flavinol and flavonol glycosides promote kidney secrete KSBGF. It follows that flavonol and flavonol glycosides can therefore be used for making medicine to promote bone formation.

In another aspect, the secreting of KSBGF from renal tubular cell was increased through stimulation by flavonol. Therefore, scientists may test the effect of the candidates on the renal tubular cell proliferation, or on the tubular cell secretion of KSBGF, or test the effect of the candidate conditioned-OKM on the osteoblast proliferation for screening medicine for promoting bone formation.

### Example 3.1

The Specification of Flavonol and Flavonol Glycosides Promoting Renal Tubular Cell Proliferation

OK cell, MC 3T3 E1 and HHF cells at 2×104/ml (0.1 ml) were added into 96 well plates, with various concentration of kaempferol was applied. After the cells were cultured for 3 days, cell growth was examined.

The rate of cell growth of MC 3T3 E1 and HHF were similar under the conditions with or without stimulation of kaempferol. OK cells proliferation after the stimulation with kaempferol was significantly increased.

The experiments indicate that compared with MC 3T3 E1 or HHF, OK cell proliferation was specifically promoted after the stimulation with flavonol and flavonol glycosides. Videlicet, flavonol and flavonol glycosides did not directly promote the osteoblast and fibroblast proliferation.

### Example 3.2

Flavonol and Flavonol Glycosides Increase the Production of KSBGF on Two Ways: Stimulate Renal Tubular Cell Secretion of KSBGF and Increase Kidney Cell Number.

Preparation of OKM and CM: OK cells at 2×10⁴/ml (0.1 ml) mixed with various concentrations of kaempferol were incubated in 96 well plate. The medium made of the same condition as above without OK cells was also incubated in the 96 well plates. After 24 hour, the mediums were collected. The OK cell growth was examined.

MC 3T3 E1 cells at 1×10⁵/ml (20 μl) were applied into 96 well plate along with OKM and CM (80 μl). After 3 days incubation, the cell numbers were examined.

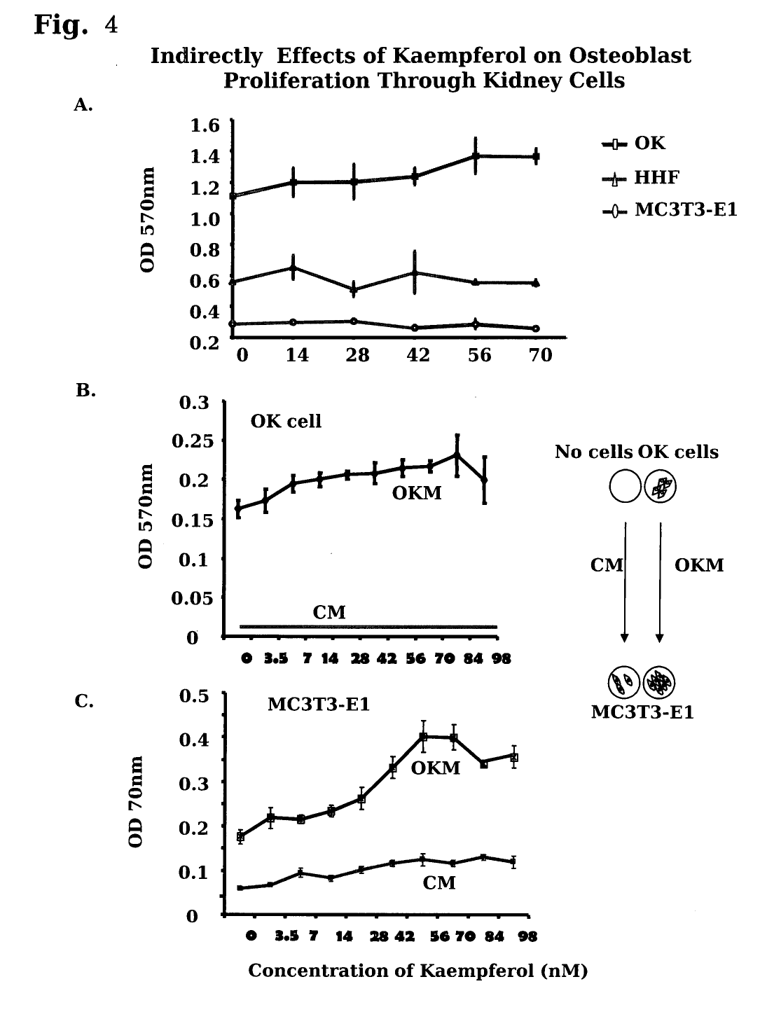

FIG. 4A shows that OK cell growth in the medium with kaempferol showed dose response aspect

FIGS. 4B and 4C show MC 3T3 E1 cells growth in the either CM with and without kaempferol are significantly lower than in the OKM. MC 3T3 E1 cell growth in the kaempferol conditioned-OKM are significantly increased compared with that in the control OKM. MC 3T3 E1 growth was not in direct ratio with the growth of OK cells.

In summary, the experiments indicate that OK cells secrete KSBGF which in turn promote MC 3T3 E1 cell proliferation (FIG. 4C). Flavonols stimulate OK cells, which significantly increase the secretion of KSBGF. This effect increases with increased concentration of kaempferol. MC 3T3 E1 proliferation in the OKM was more than expected under the assumption that the increase is in direct ratio with the presence of numbers of OK cells. It indicates that, flavonol and flavonol glycosides promote osteoblast cell proliferation indirectly through OK cell to produce KSBGF by two ways: increase OK cell number and stimulate OK cell to secrete KSBGF.

MC 3T3 E1 cell growth in both MC with or without kaempferol were similar. This indicates that the effect of flavonol and flavonol glycosides on increasing of MC 3T3 cell proliferation was not direct. Rather it was dependent on the function of OK cells.

This experiment confirmed again that OK cells secrete KSBGF and flavonol increases the secretion.

### Example 4

The Effect of KSBGF on Osteoblast Proliferation is Specially Compared with fibroblast.

The preparation of OKM and CM: OK cells at 6×10⁴/ml (3 ml) were incubated in 6 well plates over night. After the medium was removed, the fresh medium with or without 70 nM kaempferol was added. The control medium (CM) was prepared under the same condition, without OK cells. After incubation for 24 hour, all medium were collected for MC 3T3 E1 and HFF cell growth experiments.

Effect of OKM on cell proliferation in MC 3T3 and HHF:

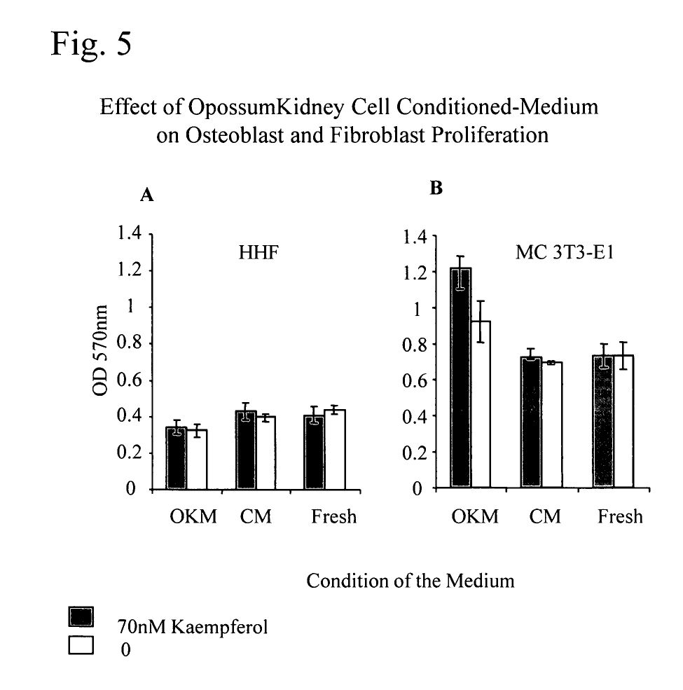

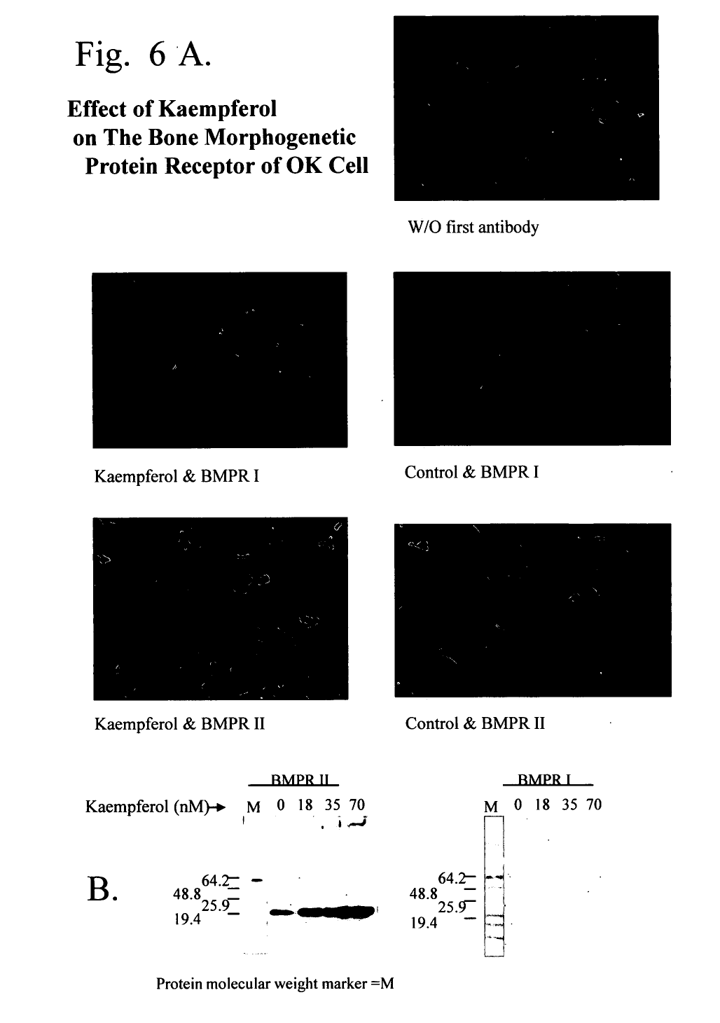

MC 3T3 E1 and HFF cell at 1×105/ml were added (20 μl) into 96 well plates. In addition, 80 μl of OKM, CM and fresh culture medium with and without kaempferol were applied. After 3 days incubation, the cell growth was examined and the results are shown in the FIGS. 5A and 5B.

FIGS. 5A and 5B show that CM and fresh medium, with and without kaempferol, did not promote MC 3T3 E1 and HHF cell proliferation. OKM promoted MC 3T3 E1 cell proliferation, but did not promote HHF cell proliferation. Kaempferol conditioned-OKM additionally promoted MC 3T3 E1 cell proliferation, and also did not promote HHF cell proliferation.

This experiment indicates that compared with HFF, the stimulation of KSBGF on osteoblast cells proliferation is restricted.

### Example 5

Flavonol and Flavonol Glycosides Increase BMPR II on OK Cell

The experiment shows BMPR I is absent on the OK cell. BMPR II is present on OK cell. Flavonol increase the density of BMPR II on OK cell.

This experiment indicates that flavonol and flavonol glycosides increase BMPR II on OK cells. Therefore, it promotes kidney cell proliferation. Flavonol and flavonol glycosides are able to be pharmaceutical used for improving kidney function and repairing kidney damage.

### Example 6

### It has Not been Repeated Yet

Flavonol and Flavonol Glycosides Stimulate OK Cell Secreting BMP 2/4, 7 and P63.

Detail of Immune-Precipitation and Detail of Western Blot Used Standard Methods and Anti-BMP 2/4 and 7.

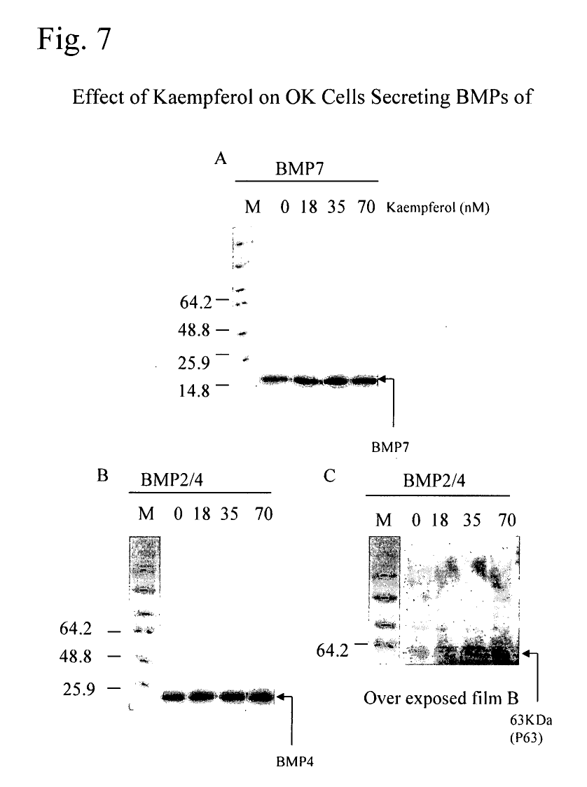

1). FIG. 7A shows that BMP7 was present in the OKM. The quantity of BMP7 was increased by dose response of kaempferol. This indicates that OK cell secreted BMP7, and kaempferol increased the ability of OK cells to secrete BMP7.

2). FIG. 7B shows that BMP2/4 was present in OKM. The quantity of BMP2/4 was increased by dose response of kaempferol. This indicates that OK cell secreted BMP2/4, and kaempferol increased the ability of OK cells to secrete BMP2/4.

3). FIG. 7C shows 63 kDa (P63) in the OKM. P63 was shown to be present after the film of 7B was over exposed. P63 was increased with dose response of kaempferol. According to the instruction from the manufacture, anti-BMP2/4 antibody may react with BMP8, 9 and 10 or other proteins. The P63 may be BMP 8, 9, 10 or another protein related to the BMP2/4.

Because the western blot is a method of specific identification of the protein which combines with the given antibody, therefore, only BMP 2/4, 7 and the molecular with the some antigenic capacity are able to show on the film. Therefore, the KSBGF, other than BMP 2/4 and 7 are not showed on this film, which also are KSBGF in this invention.

Though the above examples were to illustrate this invention, the future improvements, corrections and changes are also included in this invention under the condition that they are not left out of the spirit and essence of this invention.

### Example 7

Human Kidney Tubular Cell Conditioned Medium Promotes Osteoblast Cell Proliferation:

Human kidney tubular cells (HK-2) with the concentration at 1×10⁵/ml (1 mL) grew in 24 well plate overnight. The cells in the wells were washed by 1 mL of PBS three times. Fresh culture medium (80 μL) without FBS but contained 0 and 70 nM kaempferol were added. The same medium without cells also prepared as above. After the samples were cultured for 24 hours, the medium was collected and named respectively _{70 nm}HKM, ₀HKM, _{70 nm}CM and ₀CM. They were used for osteoblast and fibroblast culture.

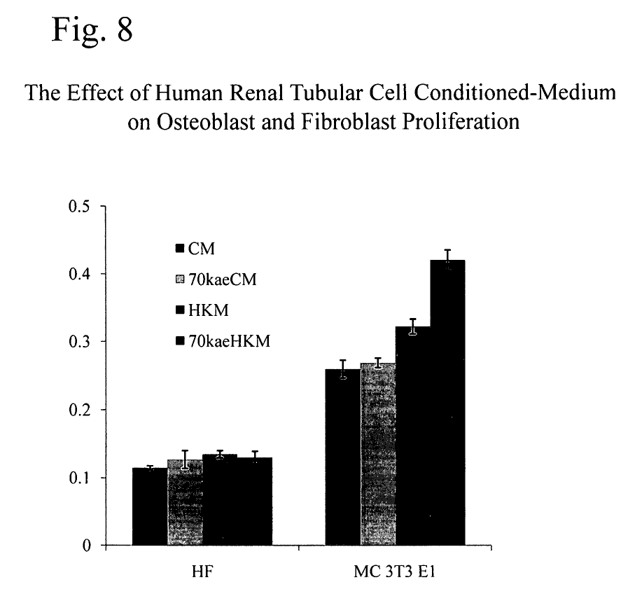

MC 3T3 E1 and HFF cell at 1×105/ml were added (20 μl) into 96 wells plate. In addition, 80 μl of _{70 nm}HKM, ₀HKM, _{70 nm}CM and ₀CM were applied. After 3 days incubation, the cell growth was examined and the results are shown in FIG. 8.

FIG. 8 shows that, MC 3T3 E1 cell grown in the ₀HKM is significantly increased than in the ₀CM, grown in the _{70 nmis}HKM is significantly increased than in the _{70 nm}CM. MC 3T3 E1 cell grown in the _{70 nmis}HKM is also significantly increased than in the ₀HKM. HKM promoted MC 3T3 E1 proliferation and kaempferol increased the promotion. The data indicates that human kidney tubular cell secretes KSBGF in turn to promote osteoblast proliferation and kaempferol increases human kidney tubular cell secretes KSBGF.

FIG. 8 also shows that HF cell grown is similar in the ₇₀ₙₘHKM, ₀HKM, _{70 nm}CM and ₀CM. Both _{70 nmis}HKM and ₀HKM have showed promoting effect on the HF proliferation. The action of human kidney tubular cell secreted KSBGF promotes osteoblast proliferation is restricted compared with HF.

Abbreviations and Acronyms

OK cell—Opossum kidney cell
CM 3T3 E1 cell—osteoblast like cell line
HF—Human fibroblast
HK-2—Human kidney tubular cell
BMP—bone morphogenetic protein
BMPR—bone morphogenetic protein receptor
OKM—Opossum kidney cell conditioned-medium
CM—Control medium
HKM—Human Kidney cell conditioned-medium

We claim:

1. A process of application of renal tubular cell secreted bone growth factor.

2. The process of claim 1, wherein said process comprising the method of examining the quantity of KSBGF that includes examining the quantity of KSBGF in body fluids or culture medium, and then using the data to analyze the capacity of kidney cells of secreting KSBGF.

3. The processes of claims 1 and 2, wherein said process includes the method for diagnosing kidney function that the process can be employed for examining the concentration of KSBGF in body fluids with the purpose of diagnosing kidney function in the secretion of KSBGF.

4. The processes of claims 1-2, wherein said process includes the process of diagnosing the state of bone formation that examines the concentration of KSBGF in the body fluids to diagnose the state of bone formation or the risk of osteoporosis.

5. The process of claim 1, wherein said that process comprising producing KSBGF, which includes incubating renal tubular cell and utilizing them to produce KSBGF.

6. The processes of claim 1, wherein said process comprises the using of KSBGF for promoting bone formation.

7. The processes of claims 1-4, wherein said that includes a method to screen medicine for treating bone formation disorder. It includes the process of the candidate being administered to contact with renal tubular cell, then examining the quantity of KSBGF in body fluids or culture medium. Moreover, this process includes analysis of the effect of the candidate on bone formation in the relation to quantity of KSBGF.

8. The processes of claims 1-7, wherein said KSBGF includes BMPs.

9. The processes of claims 1-8, wherein said KSBGF includes BMP 2, 4, 7, 8, 9 and/or 10.

10. The processes of claims 1-9, wherein said KSBGF includes BMP 2, 4, 8, 9, and/or 10 which are the antigen of BMP 2, 4, 7, 8, 9 and/or 10 and a protein has a molecular weight at about 63 kDa.

11. The processes of claims 1-7, wherein said KSBGF includes the molecules which are not the antigens of BMP 2, 4, 7, 8, 9 and/or 10.

12. A process for promoting renal tubular cells proliferation said process comprising the steps of administering an amount of flavonol or flavonol glycosides to in contact with renal tubular cells.

13. The process of claim 12, wherein said that using the composition of flavonol and flavonol glycosides in the preparation of manufacturing medicine for improving kidney function and treating renal failure.

14. The processes of claims 1, 12-13, wherein said process comprising the usage of flavonol and flavonol glycosides in the preparation of manufacturing medicine for promoting bone formation.

15. The process of claim 5, wherein said process includes a method of producing KSBGF that comprises the steps of administering the composition of flavonol and flavonol glycosides to promoting renal tubular cell proliferation.

16. The processes of claims 12-15, wherein said flavonol includes datiscetin, fisetin, kaempferol quercetin tangeretin, nobiletin, azeleatin fisetin 8-C-glucose, 5-deoxykaempferol galangin glepidotin A gossypetin, herbacetin, 6-hydrykaempferol isoquercitrin isorhamnetin kaempferide morin myricetin patuletin, quercetagetin rhamnetin robinetin and seangularetin.

17. The processes of claims 1-15, wherein said flavonol glycosides include: icariin gossypin, rutin hyperin myricitrin kaempferitrin quercitrin, quercimeritrin, and robinetin.

18. The processes of claims 1-4 and 7, wherein said body fluids include blood, tissue fluids, urine, secreted fluids and tears of humans and mammals.

19. The processes of claims 6 and 12-14, wherein said medicine is given by oral, subcutaneous, intravenous, intranasal, intracutaneous, intraperitoreal, intramusculal, intrapulmonary, intravaginal, intrarectal or intraocular routes.

20. The processes of claims 12-15, wherein said medicine includes one or more than one flavonol and flavonol glycosides together.

21. The processes of claims 5-7, 12-16 and 19, wherein said compositions include about 0.01% to about 99.99% of KSBGF or flavonol and flavonol glycosides by weight.
